(19) United States
(12) Patent Application Publication
(10) Pub. No.: US 2004/0151780 A1
(43) Pub. Date: Aug. 5, 2004

(54) Dry animal repellant

(76) Inventor: John C. Turner, Maple Valley, WA (US)

Correspondence Address: DEAN A. CRAINE, STE. 140, 400 - 112 AVE. NE, BELLEVUE, WA, 98005, US

(21) Appl. No.: 10/753,033

(22) Filed: Jan. 6, 2004

Related U.S. Application Data
(60) Provisional application No. 60/438,595, filed on Jan. 7, 2003.

(51) Int. Cl. A61K 35/14; A61K 35/78

(52) U.S. Cl. 424/529; 424/760

(57) ABSTRACT

A dry, powder, animal repellent that does not harm animals or plants. The repellant uses dried and powdered blood meal mixed with chili powder. The repellant is mixed together in a large container and then sprinkled around or into the soil around plants or into a tunnel opening formed by a rodent. In the first embodiment, designed to repel dogs and cats, the composition contains a 60%/40% (w/w) mixture of blood meal and chili powder, respectively. In the second embodiment, the concentration of blood meal to chile powder is is increased (89%/11%) to promote greater plant growth and for greater absorption into the worm and grub living in the soil.

This utility patent application is based on the provisional patent application (Serial No. 60/438,595) filed on Jan. 7, 2003.

### BACKGROUND OF THE INVENTION

1. Field of the Invention

This invention animal repellants, and more particularly, to animal repellents designed to be mixed into the soil.

2. Description of the Related Art

It is widely known that dogs and cats urinated outdoors on selected objects and plants. If the animal is allowed to urinate repeatedly at the same spot, the object and plant may be damaged or killed. A dry, premixed chemical that can be easily sprinkled around the object or plant that can be used safely with dogs and cats yet effectively repels the animal from urinating on a desired object or plant would be highly desireable.

Moles and gophers are notoriously known as rodents that form tunnels in the soil. Because mounds of dirt are generally regarded as hazards and distracting, many property owners seek various concoctions or methods to control or eradicate these rodents. Because the soil under the mound of dirt has been removed or disrupted, plants or grass located above the mound often turn brown or die.

While poisoned bait and rodent traps may be effect tools for eradicating these rodents, many property find them undesirable because of their potential to harm unintended targets such as children, dogs and cats. In order to be effective, the poisoned bait and trap must be properly placed in the tunnel or burrow so that it is not detected as a foreign obstacle. Unfortunately, even if the poisoned bait or trap is placed properly in the tunnel or burrow, small children, dogs and cats can dig from above into the tunnel and trap and disrupt the bait or trap. When the poisoned bait or trap is detected, the rodent avoids the bait and trap entirely.

What is needed is a mole or gopher repellant that can be mixed with the soil and safely used to deter moles and gophers from burrowing in a desired area. What is also needed is a repellant to small children and other small animals, that will not harm them they contact the repellent. What is also needed is a repellent that when mixed with soil, actually promotes growth of damaged plants. What is also needed is a pre-mixed repellent that can be easily dispensed by sprinkling it into the soil.

### SUMMARY OF THE INVENTION

It is an object of the present invention to provide a dry, pre-mixed animal repellant that can be easily mixed into the soil around a desired object or plant, or around a rodent's tunnel or air opening.

It is another object of the present invention to provide such a repellant that does not injury the animal or destroy the object or animal.

It is another object of the present invention to provide such a repellant that also promotes growth of earthworms to allow quick growth of the plants.

These and other objects of the present invention are met by a pre-mixed animal gopher repellant that includes powdered blood meal mixed with chili powder. In the first embodiment, designed to repel dogs and cats, the concentration of blood meal to chili powder is 60%/40% (w/w). In the second embodiment designed specifically for moles and gophers, the concentration of blood meal and chili powder is 89%/11% (w/w), respectively. The specific concentration 89%/11% (w/w) for use with moles and gopher is important so that an adequate amount of blood meal and chili is supplied to the soil to promote plant growth and still repel the rodent. The specific concentration of chili powder is also needed to irritate the rodent's mucous membranes and produce an unattractive odor when mixed into the soil. The relatively high concentration of blood meal and chili powder is also desirable because they are absorbed into worms and grubs living in the soil which moles or gophers consume and find distasteful.

When used properly, the repellant causes the dogs and cats to urinated on other objects and plants. The repellant also causes rodents to leave the existing tunnel system without killing the rodent or other animals and promotes the growth of damaged plants.

### DESCRIPTION OF THE DRAWINGS

FIG. 1 is a perspective view of a user dispensing the pre-mixed repellant from a container into a tunnel opening.

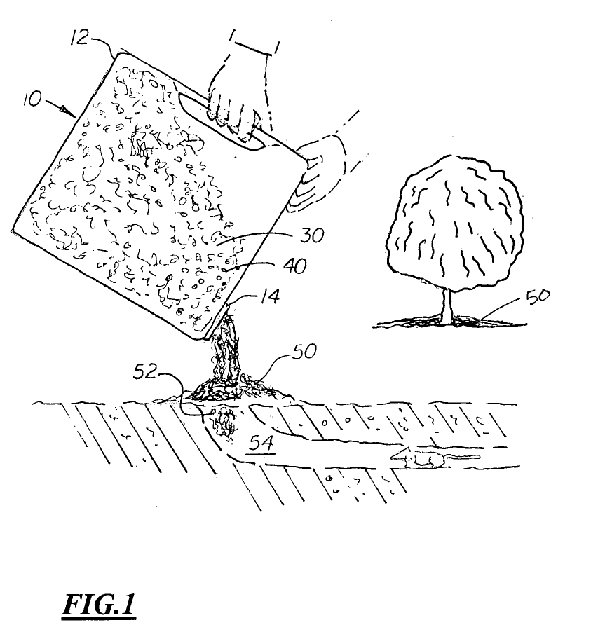

### DESCRIPTION OF THE PREFERRED EMBODIMENT(S)

There is shown and described in the accompanying Fig. an animal repellant 10 comprising powdered blood meal 30 mixed with chili powder 40. The composition 10 is pre-mixed and distributed in 1 to 2 gallon containers 12 with a large spout opening 14. During use, the composition 10 is dispensed from the container 12 and sprinkled over the top surface of soil or object to be protected or thoroughly mixed into the soil 50 around a rodent's hole 52. During use, the animal smells the blood meal 20 and chili powder 30 when urinating or when digging in the tunnel.

The particle size and specific concentration of blood meal 30 and chili powder 40 is important in order for the composition 10 to be used as a repellant for dogs and cats and rodents. In the first embodiment, the composition 10 is used as a dog and cat is a 60%/40% (w/w) mixture of blood meal 30 and chili powder 40. Both the blood meal 30 and chili powder 40 are moderately coarse to fine powders that dissolve slowly into the soil in a few days. When spread over soil, they can be easily mixed therein, yet undergo sedimentation when exposed to water.

In the second embodiment, the composition 10 is used as a mole and gopher repellant containing a 89%/11% (w/w) concentration of blood meal 30 and chili powder 40. Both the blood meal 30 and chili powder 40 are moderately coarse to fine powders that also dissolve slowly when mixed into the soil. Gradually, the blood meal 30 and chili powder 40 worms and grubs living in the soil. Such worms and grubs are distasteful to rodents which causes them to leave the existing tunnel system and find new soil.

Because blood meal 30 and chili powder 40 are not poisons to dogs, cats, and rodents, the composition 10 does not injure the animal. In compliance with the statute, the invention described herein has been described in language more or less specific as to structural features. It should be understood, however, that the invention is not limited to the specific features shown, since the means and construction shown, is comprised only of the preferred embodiments for putting the invention into effect. The invention is therefore claimed in any of its forms or modifications within the legitimate and valid scope of the amended claims, appropriately interpreted in accordance with the doctrine of equivalents.

I claim:

1. A method to repel moles and gophers, comprising:
a. selecting a mole or gopher tunnel opening;
b. mixing a rodent repellant into a large container, said repellant comprising 89% (w/w) dried and powdered blood meal mixed with 11% (w/w) chili powder;
c. sprinkling said repellant over said tunnel opening; and,
d. mixing said powdered blood meal and chili powder into the soil around said tunnel opening.

2. A mole and gopher repellant, comprising:
a. 89% (w/w), powdered blood meal; and,
b. 11% (w/w) chili powder.

3. A dog and cat repellant, comprising:
a. 60% (w/w), powdered blood meal; and,
b. 40% (w/w) chili powder.

4. An animal repellent composition, comprising:
a. 60% to 89 (w/w) powdered blood meal; and,
b. 11% to 40% (w/w) chili powder.
